(19) United States
(12) Patent Application Publication
(10) Pub. No.: US 2026/0158094 A1
(43) Pub. Date: Jun. 11, 2026

(54) COMPOSITION FOR PREVENTING, IMPROVING OR TREATING SKELETAL MUSCLE ATROPHY OR SARCOPENIA COMPRISING EXTRACT OR ISOLATED COMPOUND FROM STELLARIA DICHOTOMA AS ACTIVE INGREDIENT

(71) Applicant: INDUSTRY-UNIVERSITY COOPERATION FOUNDATION HANYANG UNIVERSITY ERICA CAMPUS, Ansan-si (KR)

(72) Inventors: Chul Young KIM, Anyang-si (KR); Jae Yong KIM, Seoul (KR); Ju Hee LEE, Incheon (KR)

(21) Appl. No.: 19/281,415

(22) Filed: Jul. 25, 2025

Related U.S. Application Data
(63) Continuation of application No. PCT/KR2024/007964, filed on Jun. 11, 2024.

(30) Foreign Application Priority Data
Jun. 13, 2023 (KR) 10-2023-0075248
Jul. 11, 2023 (KR) 10-2023-0089732

(51) Int. Cl. A61K 36/36; A61K 31/352; A61K 31/437; A61K 31/706

(52) U.S. Cl. CPC A61K 36/36; A61K 31/352; A61K 31/437; A61K 31/706; A61K 2236/33; A61K 2236/331; A61K 2236/35

(57) ABSTRACT

A pharmaceutical composition for preventing, improving, or treating skeletal muscle atrophy or sarcopenia, comprising a Stellaria dichotoma extract as an active ingredient. The composition includes the Stellaria dichotoma extract, its fractions, or one or more compounds isolated therefrom, including Dichotomine B, Glucodichotomine B, Shaftoside, or Isoshaftoside.

### CROSS-REFERENCE TO RELATED APPLICATION

This application is a bypass continuation of pending PCT International Application No. PCT/KR2024/007964, which was filed on Jun. 11, 2024, and which claims priority to and the benefit of Korean Patent Application No. 10-2023-0075248, which was filed in Korean Intellectual Property Office on Jun. 13, 2023, and Korean Patent Application No. 10-2023-0089732, which was filed in Korean Intellectual Property Office on Jul. 11, 2023, the disclosure of which are incorporated herein by reference in its entirety.

### TECHNICAL FIELD

The present invention relates to a composition for the prevention and treatment of skeletal muscle atrophy or sarcopenia, and more specifically, to a pharmaceutical composition, food composition, or feed composition for preventing, improving, or treating skeletal muscle atrophy or sarcopenia comprising an extract from Stellaria dichotoma (Stellaria dichotoma L. var. lanceolata Bunge) or a compound isolated therefrom as an active ingredient.

### BACKGROUND ART

Skeletal muscle is the largest tissue in the human body, with approximately 600 muscles comprising 40-50% of total body weight. These muscles play critical roles in physical movement, respiration, posture maintenance, body temperature regulation, and energy metabolism. The mass of skeletal muscle is regulated by a balance between protein synthesis and degradation. When degradation exceeds synthesis, muscle loss occurs. From the age of 40, muscle mass decreases by about 8% every 10 years, and after age 70, the loss accelerates to 15% per decade.

Muscle atrophy occurs due to aging, prolonged disuse, lack of physical activity, hormone deficiency, and various diseases such as cachexia, sepsis, cancer, diabetes, chronic inflammation, and degenerative disorders. Such atrophy weakens the body's basic physical strength, leads to musculoskeletal degeneration, and results in reduced mobility, diminished physical function, increased risk of falls and fractures, and overall decreased quality of life. In addition, skeletal muscle loss increases the risk of fractures, joint damage, metabolic syndrome, obesity, dyslipidemia, and cardiovascular disease.

Mechanistically, inhibition of the PI3K/Akt/mTOR pathway and its downstream factors such as 4E-BP1 and p70S6K, which leads to suppressed myotube synthesis, and increased protein degradation due to activation of the ubiquitin-proteasome pathway, are key contributors to muscle atrophy.

In contrast, regeneration of damaged skeletal muscle requires myoblast proliferation and differentiation, and myotube hypertrophic growth, which is regulated by growth factors and muscle-specific proteins such as MyoD, myogenin, and MHC (myosin heavy chain).

Many natural products such as Coptis, ginseng, black soybeans, and black ginseng have been reported to show efficacy in inhibiting muscle atrophy and promoting muscle differentiation or hypertrophy.

Stellaria dichotoma L. var. lanceolata Bunge is the dried root of a Caryophyllaceae family plant distributed in China, Russia, Europe, and Mongolia. It contains compounds such as β-carboline alkaloids, cyclic peptide alkaloids, flavonoids, neolignans, sterols, and terpenoids. Traditionally, it has been used in oriental medicine as an antipyretic and for treating wasting diseases or pediatric malnutrition.

However, until now, no studies have reported on the preventive or therapeutic effects of Stellaria dichotoma extract on skeletal muscle atrophy. Accordingly, the present inventors, as a result of diligent and extensive research, have discovered that compositions comprising Stellaria dichotoma extract exhibit excellent anti-atrophy effects, thereby completing the present invention.

### DISCLOSURE

### Technical Problem

Accordingly, the technical problem to be solved by the present invention is to provide a composition for the prevention, improvement, or treatment of skeletal muscle atrophy or sarcopenia using an extract, a fraction, or a compound isolated from Stellaria dichotoma.

### Technical Solution

To solve the above problem, the present invention provides a pharmaceutical composition for the prevention, improvement, or treatment of skeletal muscle atrophy or sarcopenia comprising Stellaria dichotoma extract as an active ingredient.

In one embodiment, the Stellaria dichotoma extract is obtained using one or more solvents selected from the group consisting of water (H₂O), lower alcohols having 1 to 4 carbon atoms, n-hexane, ethyl acetate, acetone, butyl acetate, 1,3-butylene glycol, methylene chloride, and mixtures thereof.

The composition may also include fractions obtained from the extract, or one or more compounds such as Dichotomine B, Glucodichotomine B, Shaftoside, or Isoshaftoside and their pharmaceutically acceptable salts.

The skeletal muscle atrophy treated by the composition may include one or more conditions selected from sarcopenia, disuse atrophy, mechanical unloading-induced atrophy, denervational atrophy, cachexia, drug-induced atrophy, malnutritional atrophy, and muscular dystrophy.

The extract can be prepared using conventional methods with commonly used solvents under standard temperature and pressure conditions. Preferably, the extraction may be carried out using water or a mixed solvent of water and alcohol (ethanol), for example, 75% or 100% ethanol.

The extraction methods may include hot water extraction, cold soaking, reflux extraction, or ultrasonic extraction, and the extract may be further processed by filtration, concentration, and drying (e.g., spray drying or freeze drying).

Furthermore, the extract may be fractionated using solvents such as butanol, n-hexane, methylene chloride, acetone, ethyl acetate, ether, chloroform, water, or mixtures thereof.

The extraction solvent may optionally include food-grade acids such as acetic acid, citric acid, malic acid, succinic acid, fumaric acid, tartaric acid, amino acids, or lactic acid at a concentration of 0.1 to 1% (preferably about 0.5%).

The pharmaceutical composition may be formulated into oral dosage forms (e.g., powders, granules, tablets, capsules, syrups) or parenteral forms (e.g., injections, suspensions), optionally including pharmaceutically acceptable carriers such as saline, sterile water, buffer solutions, dextrose, glycerol, ethanol, and others.

The composition may also include additional additives such as antioxidants, stabilizers, preservatives, sweeteners, flavoring agents, and emulsifiers. It can be administered orally, intravenously, subcutaneously, intramuscularly, or nasally, depending on the intended use.

The effective dosage varies depending on patient condition, age, weight, severity of disease, administration method, and other known medical parameters, and can be determined empirically by a skilled person in the field.

Additionally, the composition may be formulated into food or feed compositions using common food materials such as meat, grains, snacks, drinks, vitamins, supplements, or animal feed, offering a functional health food alternative for preventing or alleviating muscle loss.

### Advantageous Effects

The composition for preventing, ameliorating, or treating muscle atrophy or sarcopenia, comprising Stellaria dichotoma extract, fractions thereof, or Dichotomine B, Glucodichotomine B, Shaftoside, or Isoshaftoside isolated therefrom as an active ingredient, increases the thickness of muscle fibers. As it is derived from natural products, it has almost no side effects and high safety. Therefore, it can be usefully employed in the prevention, amelioration, or treatment of muscle atrophy or sarcopenia.”

### BRIEF DESCRIPTION OF DRAWINGS

FIG. 1 shows the preparation method of the extract and fractions of Stellaria dichotoma.

FIG. 2 shows the HPLC chromatographic results of the extract and fractions.

FIG. 3 shows images of C2C12 myotubes treated with the extract and fractions.

FIG. 4 shows the thickness of C2C12 myotubes after treatment with the extract and fractions.

FIG. 5 is a schematic diagram illustrating the isolation and purification process of compounds 1-5 from the n-butanol fraction of Stellaria dichotoma.

FIG. 6 shows the HPLC chromatograms of the n-butanol fraction and the isolated compounds (1-5) from Stellaria dichotoma extract.

FIG. 7 shows the chemical structures of the isolated compounds 1-5.

FIG. 8 shows images of C2C12 myotubes treated with the n-BuOH fraction and compounds 1-5.

FIG. 9 shows the degree of cell death and myotube thickness in C2C12 cells treated with 30 μg/mL n-BuOH fraction and 30 μM of compounds 1-5.

FIG. 10 shows the cell viability of 30 μM Dichotomine B (compound 2) treatment.

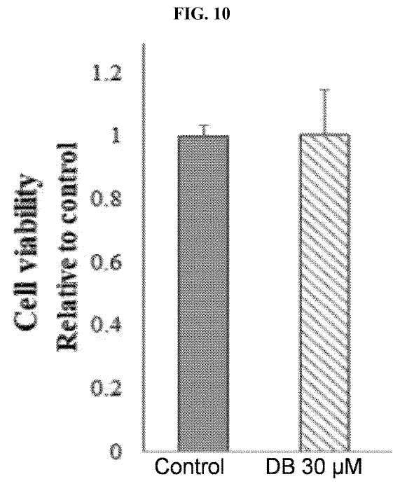

FIG. 11 shows MHC immunostaining images after treatment of Dexamethasone and 10 μM or 30 μM of Dichotomine B.

FIG. 12 shows the thickness of myotubes.

FIG. 13 shows the nuclei fusion index.

FIG. 14 shows western blot images of MHC, Atrogin-1, and MuRF-1 after Dexamethasone and Dichotomine B treatment.

FIG. 15 shows the expression levels of MHC, Atrogin-1, and MuRF-1.

FIG. 16 shows the effect of Dichotomine B at 1 μM and 10 μM on C2C12 myotubes under a starvation model, including cell viability from 1 nM to 10 μM.

FIG. 17 shows the effect of Dichotomine B on cell viability under the starvation model.

FIG. 18 shows MHC immunofluorescence images under the starvation model.

FIG. 19 shows the thickness of myotubes.

FIG. 20 shows the relative intensity of MHC staining.

FIG. 21 shows the western blot results for MHC, FoxO3a, Atrogin-1, and MuRF-1 after 1 μM and 10 μM Dichotomine B treatment under the starvation model.

FIG. 22 shows the expression level of MHC.

FIG. 23 shows the expression level of FoxO3a.

FIG. 24 shows the expression level of Atrogin-1.

FIG. 25 shows the expression level of MuRF-1.

FIG. 26 shows changes in body weight after administration of 10 mg/kg Dichotomine B in mice subjected to 48-hour starvation.

FIG. 27 shows changes in grip strength.

FIG. 28 shows muscle images of TA, EDL, GA, and SOL muscles in mice treated with 10 mg/kg Dichotomine B after 48-hour starvation.

FIG. 29 shows the weights of TA, EDL, GA, and SOL muscles.

### BEST MODE

Hereinafter, specific exemplary embodiments of the present disclosure will be described in detail referring to the attached drawings.

Prior to the description of the present disclosure, it is to be understood that the terms or words used in the present specification should not be construed as limited to their ordinary or dictionary meanings and the inventor of the present disclosure can adequately define the concepts of various terms to explain his/her invention in the best way.

In addition, it is to be understood that these terms or words should be interpreted with meanings and concepts consistent with the technical idea of the present disclosure.

That is to say, the terms used in the present specification are used only to describe the specific exemplary embodiments of the present disclosure and are not intended to specifically limit the contents of the present disclosure.

It is to be understood that the terms are defined in consideration of various possibilities of the present disclosure.

In addition, in the present specification, singular expressions may include plural expressions unless the context clearly indicates otherwise.

Similarly, it should be noted that plural expressions may also include singular expressions.

Throughout the present specification, when a certain component is described to “include” another component, any another component may exist further unless the context clearly indicates otherwise.

Furthermore, when a certain component is described to be “present in or connected to” another component, the components may be connected directly or in contact with each other.

In addition, they may be spaced apart with a certain distance. When they are spaced apart with a certain distance, there may be a third component or means for fixing or connecting the component to another component.

It is to be noted that the description of the third component or means may be omitted.

On the other hand, when a component is described to be “directly connected” to another component, it should be understood that no third component or means exists.

Likewise, other expressions that describe the relationship between components, such as “between”, “immediately between”, “neighboring”, “directly neighboring”, etc., should be interpreted as having similar meanings.

In addition, in the present specification, the terms such as “one surface”, “the other surface”, “one side”, “the other side”, the first”, “the second”, etc. are used to clearly distinguish one component from other components.

However, it should be noted that the meaning of the corresponding component is not limited by such terminology.

In addition, in the present specification, terms related to positions such as “top”, “bottom”, “left”, “right”, etc. should be understood to indicate the relative position of the corresponding component in the drawing.

In addition, unless the absolute position is specified, the position-related terms should not be understood as referring to absolute positions.

In the drawings attached to the present specification, the size, position, relationship of combination, etc. of each component may be somewhat exaggerated, reduced or omitted in order to fully convey the idea of the present disclosure or for the convenience of explanation. Therefore, the proportion or scale may be incorrect.

In addition, when describing the present disclosure, the detailed description of known technologies including prior art may be omitted to avoid unnecessarily obscuring the gist of the present disclosure.

The present invention is directed to solving the aforementioned technical problems, and as a result of extensive research to explore naturally derived substances capable of alleviating skeletal muscle loss, it was confirmed that treatment with Stellaria dichotoma extract effectively prevents the reduction in diameter of C2C12 myotubes induced by dexamethasone.

Additionally, solvent-solvent fractionation of the Stellaria dichotoma extract was performed, and the resulting fractions were also confirmed to be effective in improving muscle atrophy.

In one embodiment of the present invention, five compounds were isolated and purified from the active n-butanol fraction using various chromatographic techniques and preparative HPLC. The structures of the five compounds were identified using NMR and mass spectrometry as Glucodichotomine B, Dichotomine B, Shaftoside, Dichotomine A, and Isoshaftoside. Among these, Glucodichotomine B, Dichotomine B, Shaftoside, and Isoshaftoside effectively prevented dexamethasone-induced reduction in diameter of C2C12 myotubes.

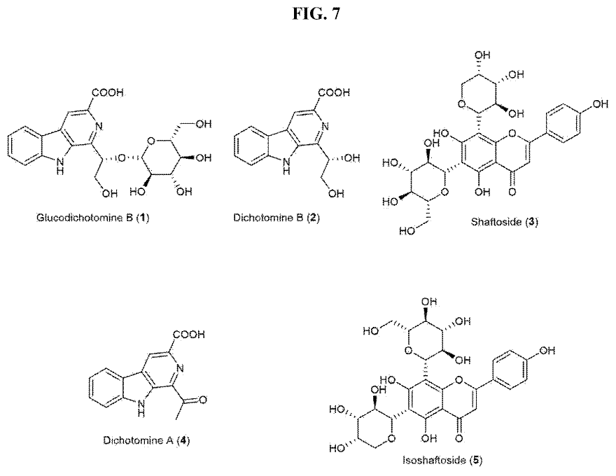

In one embodiment of the present invention, the chemical structures of Glucodichotomine B (1), Dichotomine B, Shaftoside, and Isoshaftoside are shown in FIG. 7.

In particular, the present invention identifies Dichotomine B, which was isolated and purified as an active ingredient from Stellaria dichotoma extract, as having a significant effect in preventing the reduction in diameter of C2C12 myotubes under starvation-induced atrophy models, and it also improved the MHC-stained area. Furthermore, intraperitoneal administration of 10 mg/kg Dichotomine B in 7-week-old C57BL/6 mice significantly improved muscle weight and grip strength loss caused by 48-hour starvation.

Accordingly, the present invention provides compositions for preventing, improving, or treating muscle atrophy, which comprise Stellaria dichotoma extract, fractions thereof, or isolated compounds including Dichotomine B, Glucodichotomine B, Shaftoside, or Isoshaftoside as active ingredients.

The Stellaria dichotoma extract of the present invention can be obtained using conventional methods known in the art for extracting from natural substances, that is, by extraction using common solvents under normal temperature and pressure conditions. For example, the Stellaria dichotoma extract may be obtained using one or more solvents selected from the group consisting of water, lower alcohols having 1 to 4 carbon atoms, n-hexane, ethyl acetate, acetone, butyl acetate, 1,3-butylene glycol, methylene chloride.

In a preferred embodiment, the extract may be obtained using a mixed solvent of water and a lower alcohol, and more preferably, extracted using 75% or 100% ethanol. The efficiency of extracting active ingredients with muscle atrophy improvement effects from Stellaria dichotoma may vary depending on the mixing ratio. The extraction method may be hot water extraction, cold soaking, reflux extraction, ultrasonic extraction, etc., and more preferably reflux extraction.

The obtained extract may be filtered, concentrated, and/or dried to remove the solvent. Filtration may be carried out using filter paper or vacuum filtration. Concentration may be performed under reduced pressure. Drying may be carried out by spray drying or freeze drying, but is not limited thereto. Furthermore, the extract obtained by the above method may be subjected to additional fractionation using solvents such as butanol, n-hexane, methylene chloride, acetone, ethyl acetate, ethyl ether, chloroform, water, or mixtures thereof. In addition, the present invention allows the extraction process to be carried out by adding food-grade acids to the solvent. Preferably, one or more acids selected from the group consisting of acetic acid, citric acid, malic acid, succinic acid, fumaric acid, tartaric acid, amino acids, and lactic acid may be added to the solvent. More preferably, acetic acid may be added at a concentration of 0.1 to 1% by weight, most preferably about 0.5% by weight. Accordingly, the present invention provides a method for preparing a Stellaria dichotoma extract for preventing, improving, or treating muscle atrophy, comprising: (a) pulverizing dried Stellaria dichotoma to prepare a powder; and (b) extracting the powder using one or more solvents selected from the group consisting of water, C1-C4 lower alcohols, and mixtures thereof. In a preferred embodiment, the extraction may be performed by reflux extraction, and the extract may be further concentrated under reduced pressure and freeze-dried.

In the present invention, the term “muscle atrophy” refers to a condition in which the rate of muscle degradation exceeds muscle synthesis, resulting in a progressive decrease in muscle cell number, muscle fiber area, and overall muscle mass, leading to muscle weakness and degeneration.

Muscle atrophy may be promoted by inactivity, disease, oxidative stress, and chronic inflammation, and leads to decreased muscle function and mobility. It is generally accompanied by physiological, histochemical, and biochemical changes caused by decreased muscle proteins and may result in impaired function of skeletal muscles. The Stellaria dichotoma extract of the present invention may be used to preserve the original function of skeletal muscles by enhancing and protecting muscle fibers. The sarcopenia or muscle atrophy treated by the present invention may include at least one condition selected from the group consisting of sarcopenia, muscular dystrophy, hypotonia, disuse atrophy, mechanical unloading-induced atrophy, denervational atrophy, cachexia, drug-induced atrophy, malnutritional atrophy, and other muscle degenerative diseases.

The composition may be used to prevent or improve muscle loss due to immobilization (e.g., aging, disease, prolonged bed rest, casting), peripheral nerve damage, cancer, sepsis, or long-term inactivity and aging. Atrophy due to mechanical unloading or denervation may involve reduced mitochondrial content, dysfunction, increased reactive oxygen species (ROS), oxidative damage, and muscle cell apoptosis. The composition of the present invention may be used to prevent or alleviate these symptoms.

Cachexia refers to severe weight and muscle loss seen in terminal stages of diseases like cancer, tuberculosis, and hemophilia. Muscular dystrophy refers to genetic disorders involving absence of the dystrophin-glycoprotein complex, leading to muscle atrophy and weakness. The composition may be used for prevention and symptom improvement in such cases. Cortisol, released by adrenal cortex during stress, may also cause muscle atrophy. Synthetic corticosteroids may cause steroid myopathy or muscle damage. The composition of the present invention may be useful in preventing such muscle atrophy.

### In the Present Invention:

“Prevention” refers to delaying or inhibiting the onset of muscle atrophy.
“Treatment” refers to improving or alleviating symptoms of muscle atrophy.
“Improvement” refers to reducing the severity or parameters of symptoms related to muscle loss.

The term “pharmaceutical composition” refers to preparations intended for preventing or treating disease. The composition can be formulated in various forms: powders, granules, tablets, capsules, syrups, suspensions, emulsions, suppositories, and injectable solutions.

The phrase “included as an active ingredient” means that the component is present in a necessary or sufficient amount to exhibit desired biological effects. The amount may vary based on disease state, administration route, patient condition, and is determined by a person skilled in the art. The composition may include pharmaceutically acceptable carriers such as saline, sterile water, Ringer's solution, buffers, dextrose, maltodextrin, glycerol, ethanol, and mixtures thereof. Antioxidants, stabilizers, sweeteners, and emulsifiers may also be included. Formulations may also include dispersants, surfactants, binders, lubricants, disintegrants, preservatives, and flavoring agents, depending on dosage form. Examples of oral formulations include chewable tablets, lozenges, troches, elixirs, suspensions, syrups, and wafers.

Injectable formulations may include isotonic aqueous solutions or suspensions, and may be prepared using known pharmaceutical techniques with suitable emulsifying or suspending agents. The pharmaceutical composition of the present invention may be administered orally, intravenously, subcutaneously, intramuscularly, nasally, or intraperitoneally.

A “pharmaceutically effective amount” means an amount sufficient to produce a therapeutic effect without causing side effects, depending on patient age, sex, body weight, severity of disease, and other medical considerations. The term “subject” refers to mammals including mice, rats, livestock, and preferably humans.

The composition of the present invention may also be utilized as a health functional food for the prevention or improvement of sarcopenia or muscle atrophy comprising Stellaria dichotoma extract as an active ingredient.

The term “health functional food composition” refers to a food that is produced by adding the Stellaria dichotoma extract to food ingredients such as beverages, teas, spices, gums, or confectionery, or manufactured into forms such as capsules, powders, or suspensions. These provide specific health benefits upon intake but are derived from food materials and therefore have fewer side effects compared to long-term use of conventional drugs.

Such health functional foods may be in the form of meats, grains, caffeinated drinks, general beverages, chocolates, bread, snacks, cookies, pizza, jelly, noodles, gums, ice creams, alcoholic drinks, liquors, vitamin complexes, and other dietary supplements.

The formulation can be administered in various ways including oral, transdermal, subcutaneous, intravenous, or intramuscular routes. The dosage may be appropriately selected based on the route of administration, age, gender, weight, and severity of the patient's condition.

The composition may also be co-administered with known compounds that enhance the desired effect. Administration methods may include oral (including sublingual) and parenteral routes such as subcutaneous injection, intramuscular injection, intravenous injection, and drip infusion. In addition, the pharmaceutical composition of the present invention may further include binders, lubricants, disintegrants, colorants, preservatives, sweeteners, flavoring agents, stabilizers, diluents, emulsifiers, osmotic pressure regulators, and other auxiliary agents, and may be formulated by conventional methods.

Examples of oral formulations include tablets, granules, powders, capsules, elixirs, syrups, suspensions, and wafers. The formulation may include diluents (e.g., lactose, dextrose, sucrose, mannitol, sorbitol, cellulose, and/or glycine) and excipients (e.g., silica, talc, stearic acid and its magnesium or calcium salts, and/or polyethylene glycol). Tablets may also contain binders such as magnesium aluminum silicate, starch paste, gelatin, tragacanth, methylcellulose, sodium carboxymethylcellulose, and/or polyvinylpyrrolidone, and optionally include disintegrants, absorbers, colorants, flavoring agents, and sweeteners. Such formulations may be prepared by conventional methods such as mixing, granulating, and coating. The injectable formulation may preferably be an isotonic aqueous solution or suspension and can be prepared using dispersants or wetting agents well known in the pharmaceutical field.

For parenteral administration, each component may be dissolved in saline or a buffer solution and prepared for injection. The composition of the present invention may be used alone or in combination with other functional ingredients depending on the formulation and purpose of use.

The present invention will now be described in further detail with reference to the following examples. However, it should be understood that the invention is not limited to the specific embodiments described herein, but rather may be subject to various modifications and may take on various forms. Accordingly, all variations, equivalents, and substitutions that fall within the spirit and scope of the invention are intended to be encompassed by the present disclosure. In the description of the invention, detailed explanations of well-known technologies may be omitted when they are deemed to obscure the essence of the invention.

### EXAMPLES

### Example 1. Preparation of Stellaria dichotoma Extract

Dried Stellaria dichotoma was purchased and ground using a grinder to an average particle size of 0.30 to 0.50 mm. A total of 3 kg of the pulverized material was extracted three times with 5 L of ethanol (EtOH) at 75° C. for 3 hours each. The resulting extract was concentrated using a rotary vacuum evaporator and then freeze-dried using a Heto Power Dry LL3000, yielding 366.3 g of extract.

HPLC analysis of the Stellaria dichotoma extract was performed using a Capcell Pak C18 UG120 column (5 μm, 4.6×250 mm, Shiseido, Japan). The mobile phases were: solvent A-acetonitrile and solvent B—water, both containing 0.1% formic acid. Chromatographic separation was carried out under a gradient elution at a flow rate of 1 mL/min under the following conditions:

0-5 min: 5-5% A, 5-45 min: 5-30% A, 45-85 min: 30-95% A, 85-90 min: 95-100% A

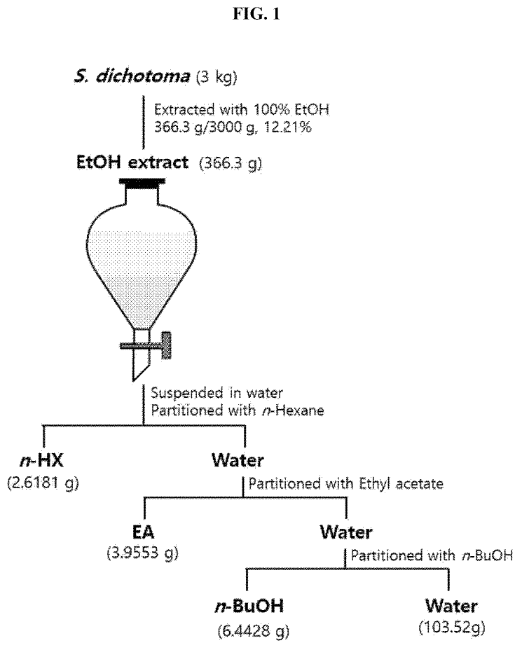

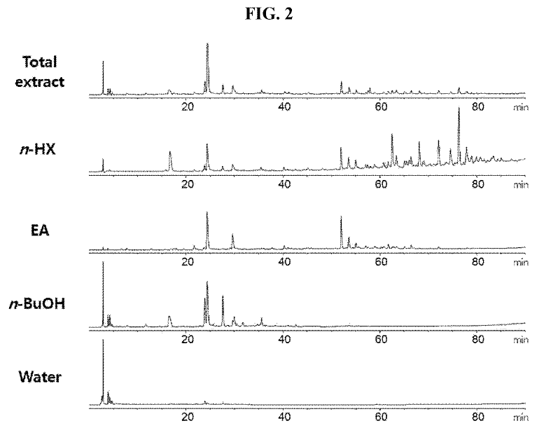

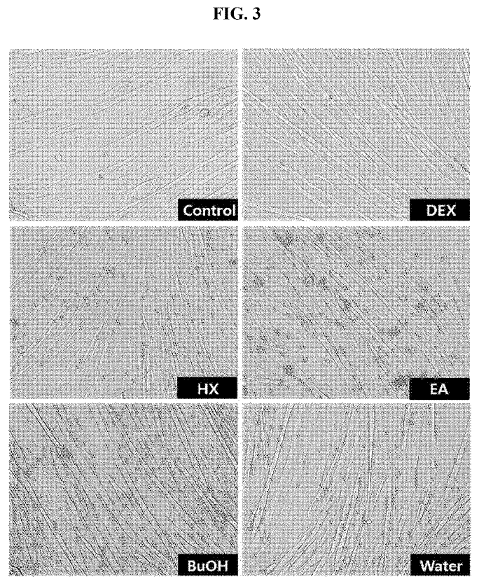

Detection was performed at 265 nm, and the results are shown in FIGS. 1 to 4.

### Example 2. Preparation of Stellaria dichotoma Fractions

To separate the extract by polarity, sequential solvent fractionation was performed using organic solvents in the following order: n-hexane (n-HX), ethyl acetate (EA), n-butanol (n-BuOH), and water. A suspension of 366.3 g of the Stellaria dichotoma ethanol extract was prepared in 1 L of water, and each solvent fractionation was repeated three times in the order of n-HX→EA→n-BuOH. Each fraction was then concentrated to obtain the respective solvent fractions.

As a result, the 366.3 g of ethanol extract was fractionated into:
2.6181 g (n-HX), 3.9553 g (EA), 6.4428 g (n-BuOH), and 103.52 g (water),

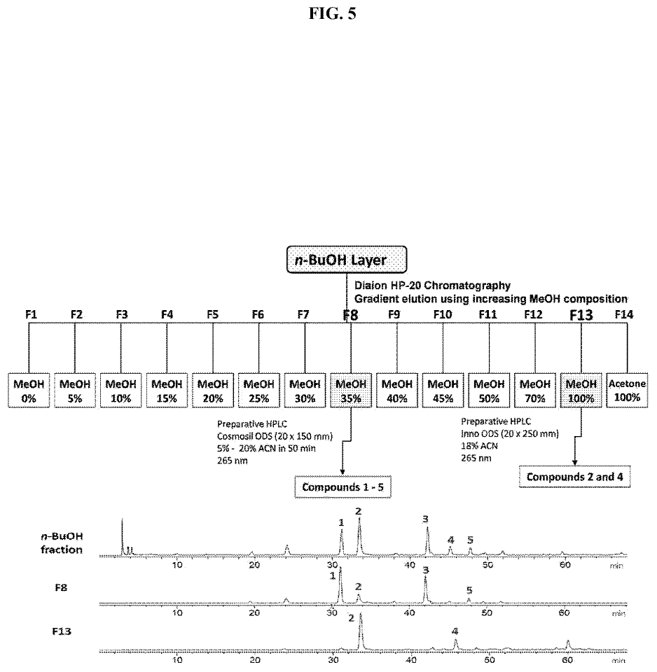

The results are shown in FIG. 5.

### Example 3. Evaluation of the Preventive, Ameliorative, or Therapeutic Effect of Stellaria dichotoma Extract or Fractions on Skeletal Muscle Atrophy In Vitro

### 3-1. Culture and Differentiation of C2C12 Cells

C2C12 mouse myoblasts used in this experiment were obtained from the American Type Culture Collection (ATCC). The cells were cultured in Dulbecco's Modified Eagle's Medium (DMEM) containing 10% fetal bovine serum (FBS), 100 U/mL penicillin, and 100 μg/mL streptomycin under conditions of 5% CO₂ at 37° C. The C2C12 cells were seeded into 12-well plates and allowed to proliferate until reaching a confluence of 85-90%.

For induction of differentiation, the culture medium was replaced with a differentiation medium composed of DMEM supplemented with 2% horse serum, 100 U/mL penicillin, and 100 μg/mL streptomycin. The medium was replaced every two days, and differentiation was carried out for a total of 6 days.

3-2. Induction of Muscle Atrophy with Dexamethasone and Treatment with Stellaria dichotoma Extract or Fractions

Dexamethasone is known to induce muscle atrophy by inhibiting protein synthesis and promoting protein degradation. In this example, C2C12 cells that had undergone differentiation as described in Example 3-1 were treated with dexamethasone to induce muscle atrophy, and subsequently treated with Stellaria dichotoma extract and/or its fractions.

The Specific Experimental Conditions were as Follows:
(1) Control Group: Differentiated C2C12 myotubes were treated with differentiation medium for 24 hours.
(2) Negative Control Group: Differentiated C2C12 myotubes were treated with differentiation medium containing dexamethasone at a final concentration of 10 μM for 24 hours.
(3) Dexamethasone+Stellaria dichotoma Extract Group: Same conditions as the negative control, with the addition of Stellaria dichotoma extract at a final concentration of 30 μg/mL for 24 hours.
(4) Dexamethasone+n-Hexane Fraction Group: Same conditions as the negative control, with the addition of n-hexane fraction of Stellaria dichotoma extract at a final concentration of 30 μg/mL for 24 hours.
(5) Dexamethasone+Ethyl Acetate Fraction Group: Same conditions as the negative control, with the addition of ethyl acetate fraction of Stellaria dichotoma extract at a final concentration of 30 μg/mL for 24 hours.
(6) Dexamethasone+n-Butanol Fraction Group: Same conditions as the negative control, with the addition of n-butanol fraction of Stellaria dichotoma extract at a final concentration of 30 μg/mL for 24 hours.
(7) Dexamethasone+Water Fraction Group: Same conditions as the negative control, with the addition of water fraction of Stellaria dichotoma extract at a final concentration of 30 μg/mL for 24 hours.

### 3-3. Measurement of C2C12 Myotube Diameter

Each control and treatment group from Example 3-2 was imaged after 24 hours of sample treatment using an automated cell imaging system (JuLi™ Stage). The diameter of the C2C12 myotubes was measured using the ImageJ cell processing software. For each experimental group, the diameters of 50 individual myotubes were measured to calculate the average and standard error.

All experiments were performed in triplicate. The results are summarized in Table 1 below.

TABLE 1

| | | DEX- | DEX + |
|---|---|---|---|
| | Control | treated group | Stellaria Extract |
| C2C12 myotube | 100 | 79.9^{a} | 96.8^{b} |
| diameter | | | |
| (% of control) | | | |

(Dex: Dexamethasone (10 μM), Stellaria dichotoma extract (30 μg/mL); ^{a}statistically significant vs. control group (p < 0.01), ^{b}statistically significant vs. DEX-treated group (negative control) (p < 0.01))

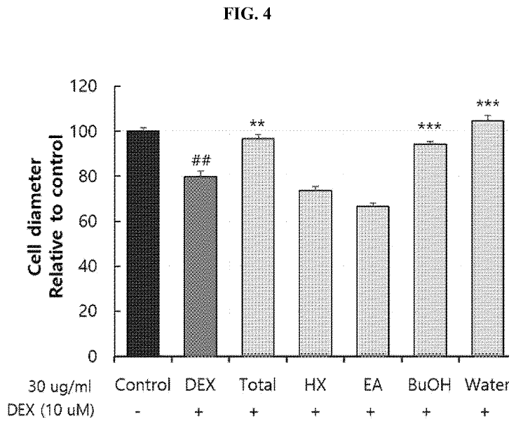

The experimental results confirmed that treatment with dexamethasone reduced the diameter of C2C12 myotubes, whereas administration of Stellaria dichotoma extract exhibited a significant ameliorative effect (see Table 1 and FIG. 4).

More specifically, the diameter of C2C12 myotubes in the group treated with dexamethasone (10 μM) was decreased by 21.1% compared to the control group treated with differentiation medium alone (p<0.01). In the group treated with both dexamethasone (10 μM) and Stellaria dichotoma extract (30 μg/mL), the diameter of C2C12 myotubes increased by 21.2% compared to the DEX-only negative control group (p<0.01). These results confirm that the Stellaria dichotoma extract of the present invention significantly restores myotube diameter in a dexamethasone-induced muscle atrophy model.

TABLE 2

| | | Dex (10 μM) treated | | | | |
|---|---|---|---|---|---|---|
| | | | n-Hexane | Ethyl Acetate | n-Butanol | Water |
| | control | | Fraction | Fraction | Fraction | Fraction |
| C2C12 | 100 | 79.9^{a} | 73.7 | 66.7 | 94.1^{c} | 104.6^{c} |
| myotube | | | | | | |
| diameter | | | | | | |
| (% of control) | | | | | | |

(Dex: Dexamethasone (10 μM), n-HX fraction: n-hexane fraction of Stellaria dichotoma extract (30 μg/mL), EA fraction: ethyl acetate fraction (30 μg/mL), n-BuOH fraction: n-butanol fraction (30 μg/mL), Water fraction: aqueous fraction (30 μg/mL))
^{a}Statistically significant vs. control group (p < 0.01);
^{c}Statistically significant vs. DEX-treated group (p < 0.001)

According to the experimental results, dexamethasone treatment led to a decrease in C2C12 myotube diameter. However, treatment with Stellaria dichotoma extract or its fractions resulted in a significant improvement (see Table 2 and FIG. 4). More specifically, (1) Myotube diameter in the dexamethasone-treated group decreased by 21.1% compared to the control group (p<0.01); (2) Myotube diameter in the n-butanol fraction-treated group increased by 17.8% compared to the DEX-only group (p<0.001); (3) Myotube diameter in the aqueous fraction-treated group increased by 30.9% compared to the DEX-only group (p<0.001).

These results confirm that the n-butanol and aqueous fractions of Stellaria dichotoma extract significantly restore C2C12 myotube diameter in dexamethasone-induced atrophy models.

### Example 4. Isolation, Purification, and Structure Elucidation of Compounds 1-5 from the n-Butanol Fraction of Stellaria dichotoma

The biologically active n-butanol (n-BuOH) fraction of Stellaria dichotoma extract was subjected to open column chromatography using Diaion HP-20 macroporous resin. Methanol (MeOH) was used as the mobile phase, with the MeOH concentration gradually increased from 0% to 100% in 5% increments. A total of 1 L of each MeOH concentration was passed through the column three times. The column was then washed three times with 1 L of acetone. The solvents were removed using rotary vacuum evaporation and freeze drying. Among the 14 initial fractions obtained, fractions 8 and 13 were further purified using preparative HPLC. Separation was performed at 265 nm, the absorbance maximum for β-carboline alkaloids, using a mobile phase of acetonitrile (ACN) and water containing 0.1% trifluoroacetic acid (TFA). Fraction 8 was applied to a Cosmosil C18 column (20× 150 mm) and eluted under a gradient condition of 5-20% ACN over 50 minutes, yielding Compounds 1-5. Fraction 14 was applied to an INNO C18 column (20× 250 mm) and eluted under isocratic conditions with 18% ACN to isolate Compounds 2 and 4 (see FIG. 5).

### Example 5. Structure Elucidation of Compounds Isolated and Purified from Stellaria dichotoma

The isolated compounds were structurally characterized using spectroscopic methods including NMR and mass spectrometry.

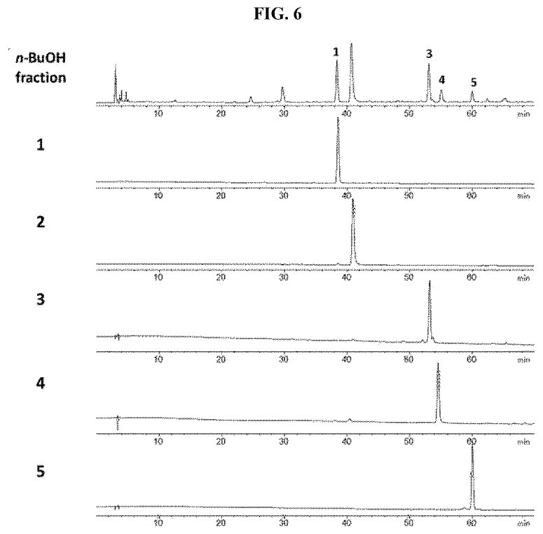

The five compounds were identified as Glucodichotomine B (Compound 1), Dichotomine B (Compound 2), Shaftoside (Compound 3), Dichotomine A (Compound 4), and Isoshaftoside (Compound 5) (see FIGS. 6 and 7).

### Glucodichotomine B

¹H NMR (400 MHz, DMSO-d₆): δ ppm 4.05 (2H, d, J=7.9 Hz, H-15), 4.52 (1H, d, J=7.6 Hz, G-1′), 5.42 (1H, m, H-14), 7.42 (1H, t, H-6), 7.62 (1H, t, H-7), 7.71 (1H, d, J=7.4 Hz, H-8), 8.40 (1H, d, J=7.4 Hz, H-5), 8.87 (1H, s, H-4), 11.79 (1H, s, NH-9).

¹³C NMR (100 MHz, DMSO-d₆): δ ppm 61.71 (G-6′), 64.95 (C-15), 70.63 (G-4′), 74.32 (G-2′), 76.88 (G-5′), 77.24 (C-14), 77.59 (G-3′), 102.00 (G-1′), 112.95 (C-8), 116.96 (C-4), 120.59 (C-6), 121.10 (C-12), 122.43 (C-5), 129.06 (C-11), 129.36 (C-7), 135.74 (C-10), 136.74 (C-3), 141.45 (C-13), 143.21 (C-1), 167.79 (C-16)

MS (ESI, positive) m/z=435.2 [M+H]⁺, (ESI, negative) m/z=433.2 [M−H]⁻

UV (MeOH) λmax=236 nm, 269 nm

### Dichotomine B

¹H NMR (400 MHz, DMSO-d₆): δ ppm 3.85 (2H, d, J=7.9 Hz, H-15), 5.19 (1H, m, H-14), 7.31 (1H, t, H-6), 7.60 (1H, t, H-7), 7.75 (1H, d, J=7.5 Hz, H-8), 8.39 (1H, d, J=7.5 Hz, H-5), 8.85 (1H, s, H-4), 11.83 (1H, s, NH-9).

¹³C NMR (100 MHz, DMSO-d₆): δ ppm 66.85 (C-15), 73.90 (C-14), 113.19 (C-8), 116.63 (C-4), 120.62 (C-6), 121.15 (C-12), 122.46 (C-5), 129.13 (C-11), 129.13 (C-7) 135.31 (C-10), 135.51 (C-3), 141.66 (C-13), 145.69 (C-1), 166.82 (C-16).

MS (ESI, positive) m/z=273.2 [M+H]⁺, MS (ESI, negative) m/z=271.2 [M−H]⁻

UV (MeOH) λmax=236 nm, 269 nm

### Schaftoside

¹H NMR (400 MHz, DMSO-d₆): δ ppm 4.53 (1H, d, J=8.7 Hz, G-1′), 4.96 (1H, d, J=9.3 Hz, A-1′), 6.81 (1H, s, H-3), 6.95 (2H, d, J=8.0 Hz, H-3′, 5′), 7.98 (2H, d, J=8.0 Hz, H-2′, 6′), 13.80 (1H, s, OH-5)

¹³C NMR (100 MHz, DMSO-d₆): δ ppm 61.32 (G-2), 66.92 (G-6), 70.31 (A-2), 70.49 (A-4), 70.79 (A-5, G-4), 74.22 (A-3), 74.77 (G-1), 75.42 (A-1), 79.88 (G-3, G-5), 108.08 (C-3), 108.54 (C-10), 104.20 (C-8), 109.83 (C-6), 116.47 (C-3′, 5′), 121.80 (C-1′), 129.24 (C-2′,6′), 153.89 (C-9), 161.70 (C-4′), 164.11 (C-5, C-7), 182.88 (C-4).

MS (ESI, positive) m/z=565.3 [M+H]⁺, MS (ESI, negative) m/z=563.3 [M−H]⁻

UV (MeOH) λmax=270 nm, 330 nm

### Dichotomine A

¹H NMR (400 MHz, DMSO-d₆): δ ppm 1.58 (3H, d, J=7.2 Hz, H-15), 5.23 (1H, m, H-14), 7.26 (1H, t, H-6), 7.60 (1H, t, H-7), 7.74 (1H, d, J=7.0 Hz, H-8), 8.37 (1H, d, J=7.0 Hz, H-5), 8.81 (1H, s, H-4), 11.83 (1H, s, NH-9)

¹³C NMR (100 MHz, DMSO-d₆): δ ppm 25.04 (C-15), 64.80 (C-14), 113.14 (C-8), 116.27 (C-4), 120.52 (C-6), 121.29 (C-12), 122.34 (C-5), 128.63 (C-7), 128.70 (C-11), 129.37 (C-10), 129.61 (C-3), 137.91 (C-13), 141.56 (C-1), 169.20 (C-16).

MS (ESI, positive) m/z=257.2 [M+H]⁺, MS (ESI, negative) m/z=255.2 [M−H]⁻

UV (MeOH) λmax=237 nm, 269 nm

### Isoschaftoside

¹H NMR (400 MHz, DMSO-d₆): δ ppm 4.72 (1H, d, J=8.5 Hz, G-1′), 4.78 (1H, d, J=9.1 Hz, A-1′), 6.83 (1H, s, H-3), 6.95 (2H, d, J=7.9 Hz, H-3′, 5′), 7.95 (2H, d, J=7.9 Hz, H-2′, 6′), 13.69 (1H, s, OH-5)

¹³C NMR (100 MHz, DMSO-d₆): δ ppm 61.25 (G-6), 68.88 (G-2), 70.96 (A-2), 71.07 (A-4), 74.77 (A-3, G-1), 74.99 (A-1), 79.17 (G-3), 79.48 (G-5), 108.23 (C-3), 104.13 (C-10), 104.95 (C-8), 109.67 (C-6), 116.40 (C-3′, 5′), 121.93 (C-1′), 129.11 (C-2′, 6′), 156.28 (C-9), 160.84 (C-4), 161.71 (C-5, C-7), 163.51 (C-2), 182.76 (C-4).

MS (ESI, positive) m/z=565.3 [M+H]⁺, MS (ESI, negative) m/z=563.3 [M−H]⁻

UV (MeOH) λmax=270 nm, 330 nm

### Example 6. Evaluation of the Preventive, Ameliorative, or Therapeutic Effect of Compounds 1-5 Isolated from Stellaria dichotoma on Skeletal Muscle Atrophy

### 6-1. Culture and Differentiation of C2C12 Cells

The same experimental methods as described in Example 3-1 were used.

6-2. Induction of Muscle Atrophy with Dexamethasone and Treatment with Compounds 1-5 Isolated from Stellaria dichotoma

The experiment was conducted in a manner similar to Example 3-2. However, instead of the extract or fraction, each of Compounds 1 to 5 was treated at a concentration of 30 μM under similar conditions.

### 6-3. Measurement of C2C12 Myotube Diameter

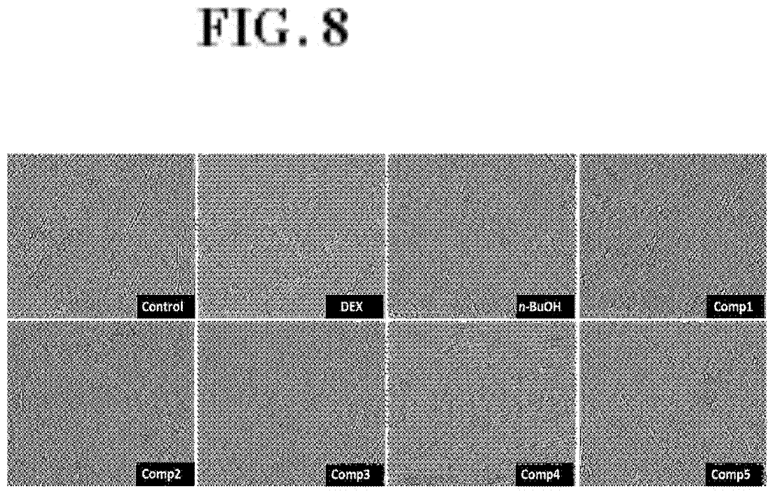

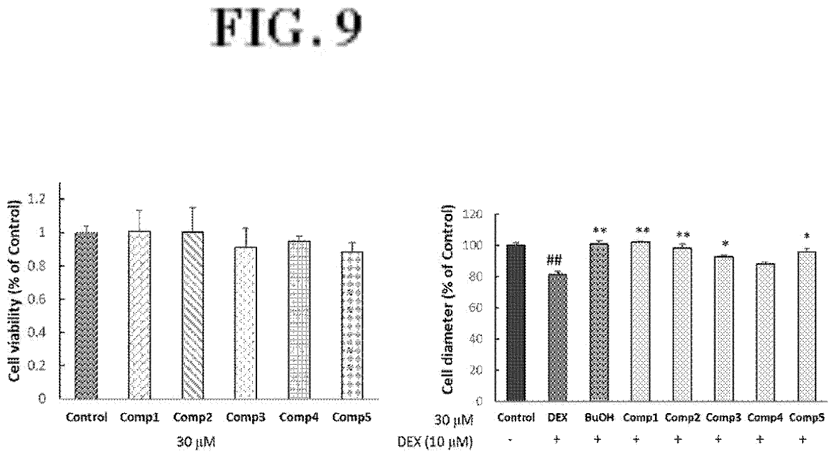

The diameter of the cells was measured using the same method described in Example 3-3. The average and standard error were calculated and the data were compiled (see Table 3 and FIGS. 8 and 9). Each experiment was performed in triplicate.

TABLE 3

| | | Dex (10 μM) Treated | | | | | | |
|---|---|---|---|---|---|---|---|---|
| | | | n-BuOH | Glucodichotomine | Dichotomine | Shaftoside | Dichotominee | Isoshaftoside |
| | | | Fraction | B (1) | B (2) | (3) | A (4) | (5) |
| | Control | — | (30 μg/mL) | 30 μM | 30 μM | 30 μM | 30 μM | 30 μM |
| C2C12 | 00.0 | 1.3^{a} | 100.7^{b} | 102.0^{b} | 98.2^{b} | 92.7^{c} | 87.9 | 95.7^{c} |
| myotube | | | | | | | | |
| diameter | | | | | | | | |
| (% of | | | | | | | | |
| control) | | | | | | | | |

(Dex: Dexamethasone (10 μM);
^{a}Statistically significant vs. control group (p < 0.01);
^{b}Statistically significant vs. DEX-treated group (p < 0.01);
^{c}Statistically significant vs. DEX-treated group (p < 0.05))

The experiments confirmed that treatment with dexamethasone reduced the diameter of C2C12 myotubes, and compounds 1-5, which were isolated, purified, and structurally identified from Stellaria dichotoma, showed improvement effects (see Table 3 and FIGS. 8 and 9).

### Example 7. Evaluation of the Ameliorative or Therapeutic Effect of Compound 2 (Dichotomine B) Isolated from Stellaria dichotoma on Skeletal Muscle Loss

### 7-1. Culture and Differentiation of C2C12 Cells

The same experimental methods as described in Example 3-1 were used.

7-2. Induction of Muscle Atrophy with Dexamethasone and Treatment with Dichotomine B Isolated from Stellaria dichotoma

The experiment was conducted similarly to Example 3-2. However, Dichotomine B was administered at concentrations of 10 μM and 30 μM.

### 7-3. Immunofluorescence Staining of Myosin Heavy Chain (MHC) in C2C12 Myotubes

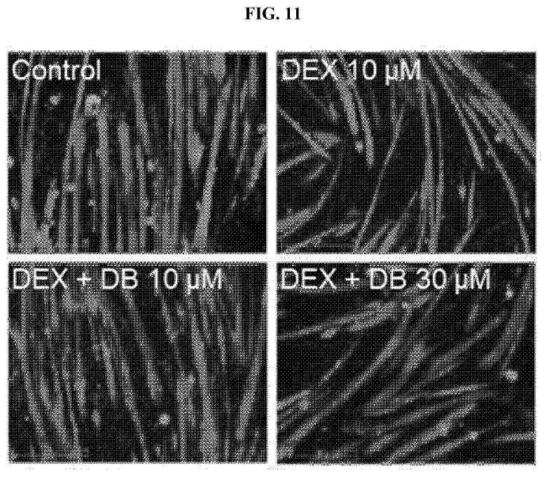

After completion of treatment, the culture media from each group was removed, and cells were washed twice with phosphate-buffered saline (PBS), fixed with 4% paraformaldehyde for 15 minutes, and washed three times with PBS. The cells were then permeabilized with 0.1% Triton X-100 for 10 minutes, washed twice with PBS, and blocked with 3% horse serum solution for 2 hours. Mouse anti-MHC antibody was diluted 1:100 in 3% BSA and incubated overnight at 4° C. The cells were washed three times with 0.1% PBST. Goat anti-mouse IgG-FITC secondary antibody (Thermo Fisher Scientific) was diluted 1:500 in 3% BSA and reacted for 1 hour at 37° C., followed by three washes with 0.1% PBST. Next, cells were stained with 1 μg/mL Hoechst 33342 solution for 10 minutes at room temperature. Cell imaging was performed using the JuLi™ system. MHC immunofluorescence was visualized in green, and cell nuclei stained with Hoechst 33342 were visualized in blue. Quantitative analysis was performed using ImageJ software, and the results are shown in FIG. 11.

### 7-4. Analysis of Fusion Index of Multinucleated Myotubes

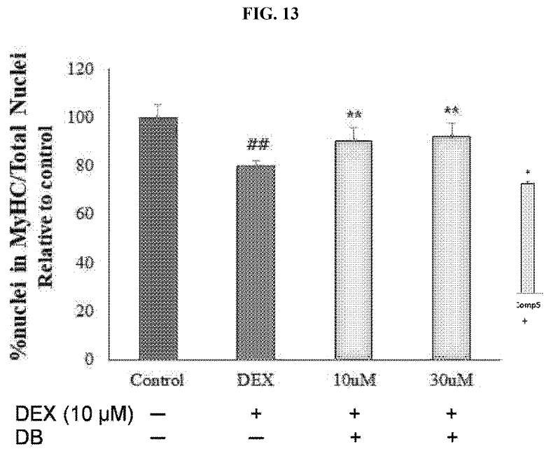

From the results of Section 7-3, randomly selected multinucleated myotubes were analyzed to calculate the fusion index. The number of multinucleated myotubes (i.e., myotubes containing multiple nuclei) was counted and normalized to the total number of cells. The relative number of MHC-positive multinucleated myotubes was determined for each treatment group (see Table 4 and FIG. 13).

TABLE 4

| | | Dex (10 μM) treated group | | |
|---|---|---|---|---|
| | | | Dichotomine | Dichotomine |
| | | | B (2) | B (2) |
| | Control | — | 10 μM | 30 μM |
| C2C12 myotube | 100.0 | 80.0^{a} | 90.3^{b} | 92.3^{b} |
| diameter | | | | |
| (% of control) | | | | |

Dex: Dexamethasone (10 μM); ^{a}Statistically significant vs. control group (p < 0.01); ^{b}Statistically significant vs. DEX-treated group (p < 0.01))

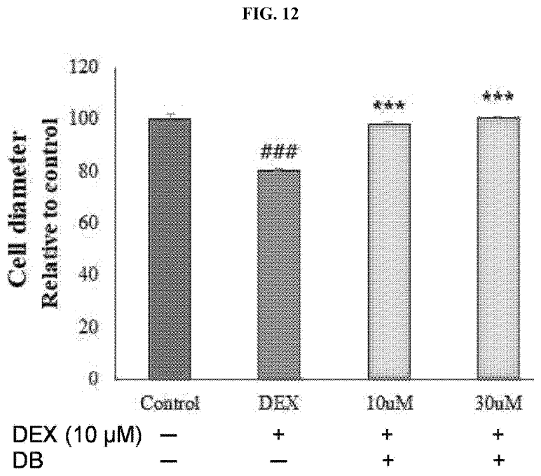

The experiments confirmed that treatment with dexamethasone reduced the fusion index of multinucleated C2C12 myotubes, whereas Dichotomine B (Compound 2), which was isolated, purified, and structurally identified from Stellaria dichotoma, significantly improved the fusion index (see Table 4 and FIGS. 12 and 13).

### 7-5. Measurement of C2C12 Myotube Diameter

The diameter of C2C12 myotubes was measured in the same manner as described in Example 3-3. The mean and standard error values were calculated and organized in Table 5. Each experiment was performed in triplicate.

TABLE 5

| | | Dex (10 μM)treated group | | |
|---|---|---|---|---|
| | | | Dichotomine | Dichotomine |
| | | | B (2) | B (2) |
| | control | — | 10 μM | 30 μM |
| C2C12 myotube | 100.0 | 80.0^{a} | 97.9^{b} | 100.6^{b} |
| diameter | | | | |
| (% of control) | | | | |

(Dex: Dexamethasone (10 μM); ^{a}Statistically significant vs. control group (p < 0.01); ^{b}Statistically significant vs. DEX-treated group (p < 0.01))

The experiments confirmed that dexamethasone treatment led to a reduction in the thickness of C2C12 myotubes, whereas Dichotomine B (Compound 2), which was isolated, purified, and structurally identified from Stellaria dichotoma, exhibited a significant ameliorative effect (see Table 5).

### 7-6. Western Blotting

C2C12 cells were seeded in a 6-well plate at a density of 1×10⁵ cells per well. After differentiation was complete, each group was treated with dexamethasone and the test sample according to the respective experimental condition.

Cells were lysed using a lysis buffer containing LIPA buffer, protease inhibitors, and phosphatase inhibitors. A volume of 200 μL of the prepared lysis buffer was added per well, and the cells were collected using a cell scraper. The lysates were centrifuged at 13,000 rpm for 20 minutes, and the supernatants were collected. Protein concentrations were measured using the BCA protein assay method. Equal amounts of protein were separated using 8% SDS-PAGE.

The separated proteins were transferred to a nitrocellulose membrane, followed by blocking with 3% BSA buffer. The membranes were incubated with various primary antibodies (e.g., MHC, FoxO3a, MuRF1, GAPDH), followed by sequential washing, incubation with secondary antibodies, and further washing steps.

Protein expression was detected using enhanced chemiluminescence (ECL), and images were acquired using the ChemiDoc imaging system. Quantification of each protein expression level was performed using ImageJ software.

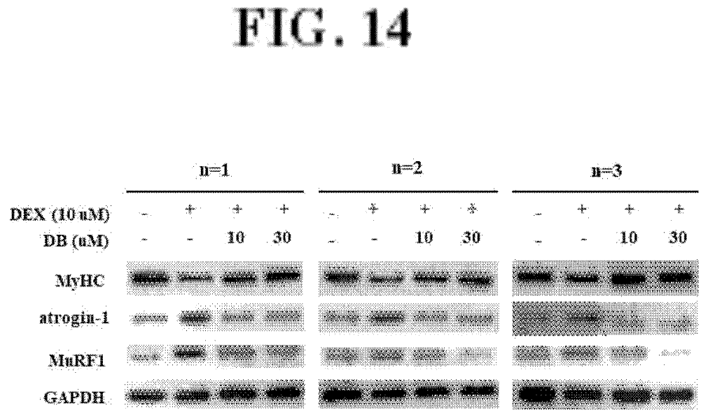

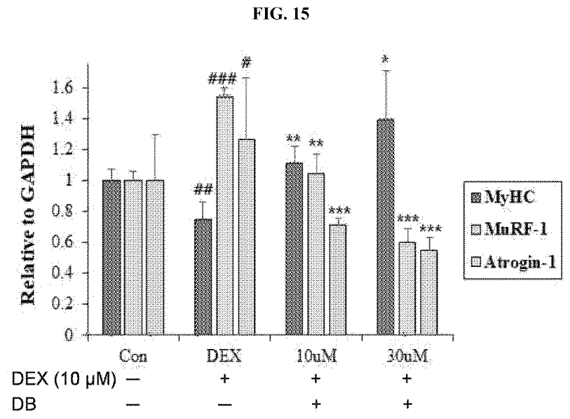

The experiments confirmed that dexamethasone treatment significantly increased the expression of Atrogin-1 and MuRF-1 while decreasing MHC in C2C12 myotubes. Upon treatment with Dichotomine B (Compound 2), MHC expression increased in a concentration-dependent manner, while the elevated levels of Atrogin-1 and MuRF-1 were reduced (see FIGS. 14 and 15).

### Example 8. Evaluation of the Preventive, Ameliorative, or Therapeutic Effect of Dichotomine B Isolated from Stellaria dichotoma in a Starvation-Induced Skeletal Muscle Atrophy Model

### 8-1. Culture and Differentiation of C2C12 Cells

The same experimental method as described in Example 3-1 was used.

8-2. Treatment with Dichotomine B in an In Vitro Starvation Model

Culturing differentiated C2C12 myotubes in serum-free/low-glucose medium inhibits muscle protein synthesis and induces muscle atrophy through increased protein degradation. In this example, C2C12 myotubes differentiated as in Section 8-1 were cultured in serum-free/low-glucose medium to induce atrophy, and the cells were then treated with Dichotomine B under the following specific experimental conditions:
1. Control group: Differentiated C2C12 myotubes were maintained in normal differentiation medium for 48 hours.
2. Negative control group: Differentiated C2C12 myotubes were cultured in serum-free/low-glucose medium for 48 hours.
3 Dichotomine B treatment groups: In the same condition as the negative control group, Dichotomine B was additionally administered at concentrations of 1 μM or 10 μM for 48 hours.

### 8-3. Immunofluorescence Staining of MHC in C2C12 Myotubes

This procedure was conducted in the same manner as described in Section 7-3.

### 8-4. Measurement of C2C12 Myotube Diameter and Analysis of MHC-Stained Area

The diameter and staining area of the myotubes were measured as described in Example 3-3. The mean and standard errors were calculated, and the results were organized into Table 6. Each experiment was performed in triplicate.

TABLE 6

| | | Starvation (Serum free/Low glucose medium)) | | |
|---|---|---|---|---|
| | | | Dichotomine | Dichotomine |
| | | | B (2) | B (2) |
| | Control | — | 1 μM | 10 μM |
| C2C12 myotube | 100.0 ± 9.2 | 80.9 ± 8.2^{a} | 98.6 + 9.3^{b} | 100.4 ± 10.8^{b} |
| diameter | | | | |
| (% of control) | | | | |
| MHC positive area | 100 ± 8.1 | 87.1 ± 5.4^{a} | 93.6 + 7.8^{b} | 97.1 ± 8.4^{b} |
| (% of control) | | | | |

(Dex: Dexamethasone (10 μM); ^{a}Statistically significant vs. control group (p < 0.01); ^{b}Statistically significant vs. DEX-treated group (p < 0.01))

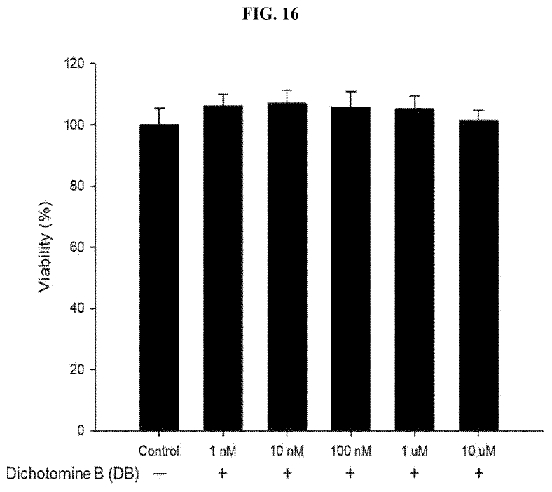

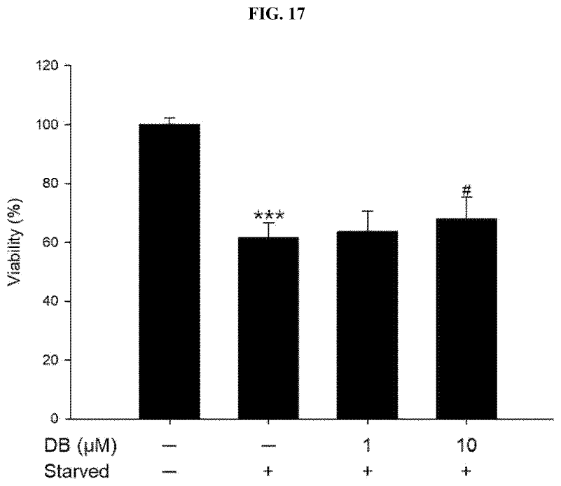

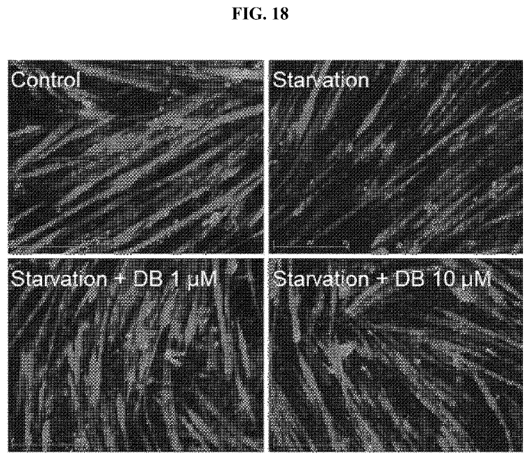

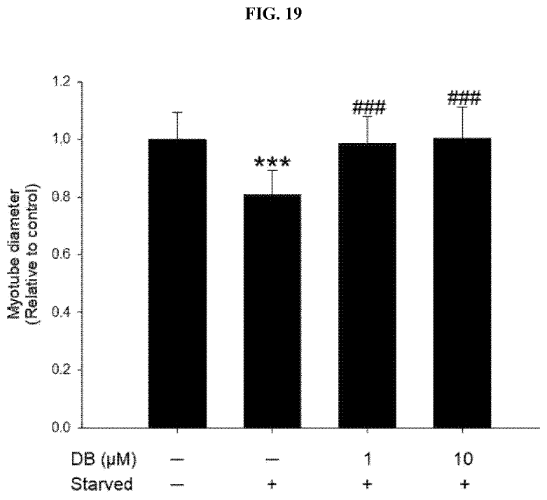

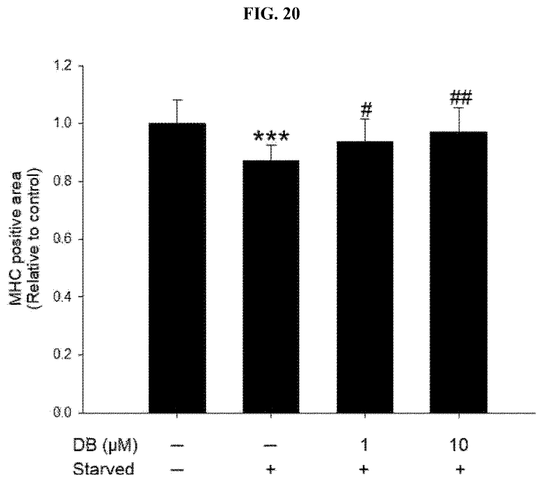

In the above experiment, it was confirmed that Dichotomine B significantly increased both the diameter of myotubes and the area of immunofluorescence detected using MHC staining (see Table 6 and FIGS. 16 to 20).

### 8-5. Analysis of Muscle Atrophy-Related Protein Expression

Western blotting was performed in the same manner as described in Example 7-5 to assess the expression levels of related proteins.

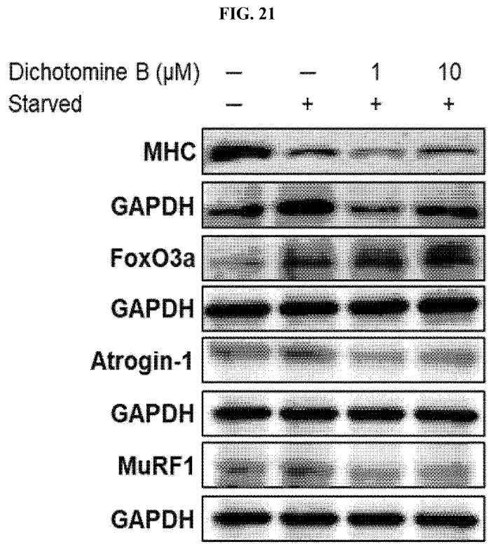

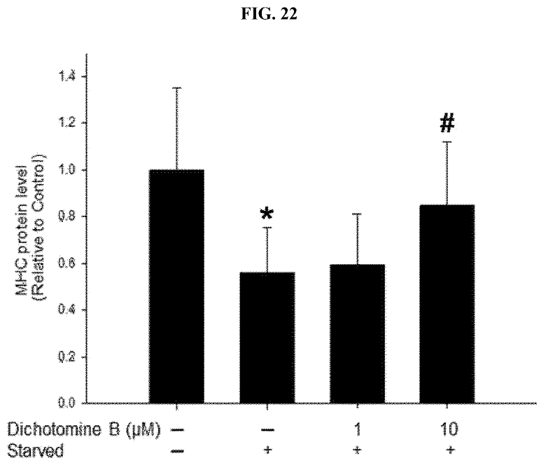

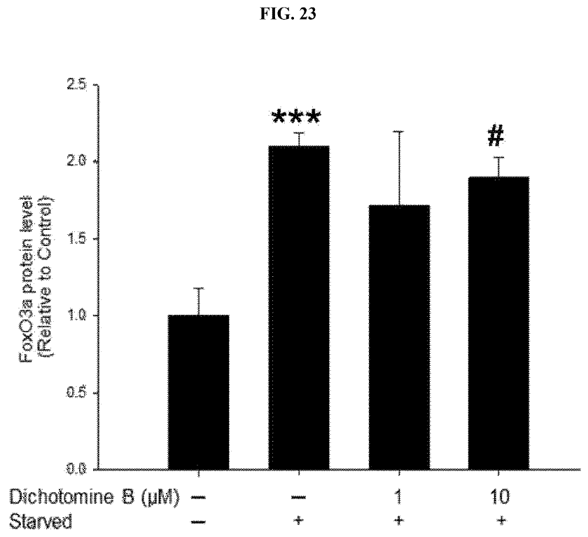

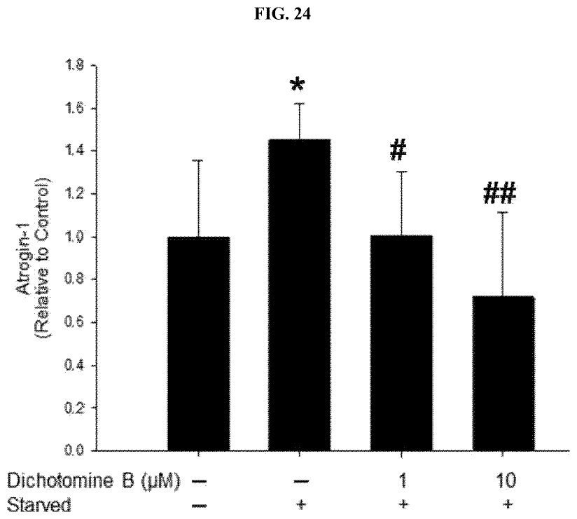

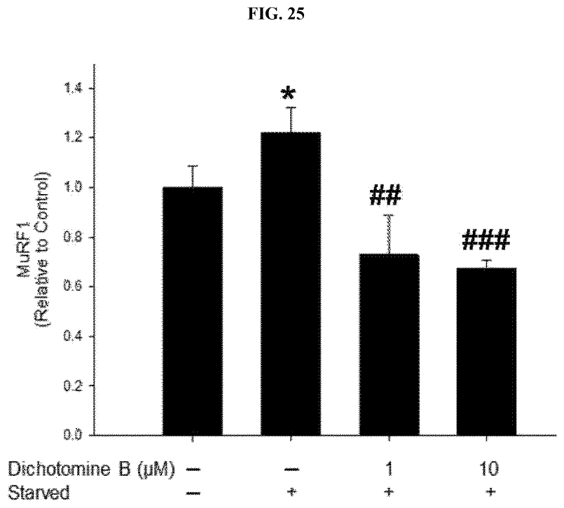

The experiments confirmed that in the C2C12 starvation model, the expression of MHC was reduced, while FOXO3a, Atrogin-1, and MuRF-1 were significantly increased. Treatment with Dichotomine B effectively reversed these effects (see FIGS. 21 to 25).

### Example 9. In Vivo Evaluation of the Preventive and Ameliorative Effects of Dichotomine B on Muscle Atrophy

### 9-1. Experimental Animals

C57BL/6 male mice (6 weeks old) were obtained from Koatech (Pyeongtaek, Gyeonggi-do, Korea) and acclimated for one week in the Hanyang University ERICA Laboratory Animal Center (Animal Facility Registration No. 445) before use in the experiments. The lighting cycle was set to 12 hours (07:00 to 19:00), and food and water were provided ad libitum. All animal experiments were conducted in compliance with the guidelines and regulations of the Institutional Animal Care and Use Committee (IACUC) of Hanyang University.

### 9-2. Induction of Muscle Loss Model and Administration of Test Sample

After acquisition of the experimental animals and a 7-day acclimation period, the experiment was conducted.

Solid feed for laboratory animals (Woojung Bio, Hwaseong, Gyeonggi-do, Korea) was provided, and all treatments were administered during a fixed time window (10:00 AM to 12:00 PM).

Body weight was measured three times in total. For the fasting group, each animal was housed individually during the fasting period. The test sample was administered twice: once at the start of the fasting period, and again 24 hours later. Experimental results are presented as means±standard deviation (SD). Statistical significance among groups was analyzed using ANOVA t-tests via the StatView program.

### 9-3. Assessment of Body Weight Changes

In the non-fasting group, body weight changes were within the normal range. However, in the fasting group (F group), a statistically significant reduction in body weight was observed after 48 hours of fasting (see Table 7).

TABLE 7

Body weight (g)

| | Start of Sample | End of Sample |
|---|---|---|
| Group | Administration | Administration |
| Feed | 21.167 ± 0.339 | 21.950 ± 0.404 |
| Feed + | 21.080 ± 0.370 | 21.400 ± 0.339 |
| Dichotomine B | | |
| Starvation | 21.800 ± 0.666 | 18.028 ± 1.254*** |
| Starvation + | 22.214 ± 0.701 | 17.743 ± 0.885*** |
| Dichotomine B | | |

(***p < 0.001 vs. Feed group)

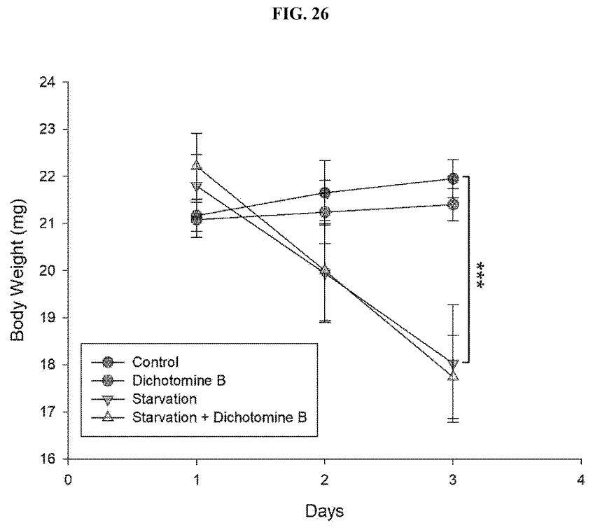

The experimental results confirmed that a 48-hour fasting period induced a reduction in body weight in the mice used in the study, while administration of Dichotomine B had no significant effect on body weight (see Table 7 and FIG. 26).

### 9-4. Evaluation of Grip Strength

After the completion of the fasting period, grip strength was measured using a grip strength meter.

TABLE 8

| Group | Grip Strength (kgf) |
|---|---|
| Feed | 192.100 ± 6.973 |
| Feed + | 190.400 ± 11.747 |
| Dichotomine B | |
| Starvation | 154.393 ± 12.630*** |
| Starvation + | 187.114 ± 10.724^{###} |
| Dichotomine B | |

(***p < 0.001 vs. Feed group, ^{###}p < 0.001 vs. Starvation group)

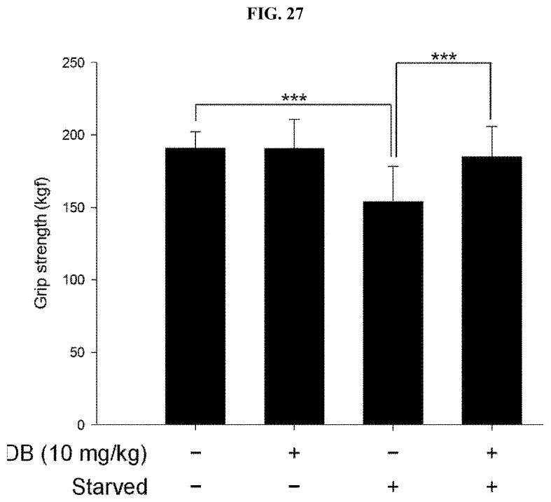

The experimental results confirmed that 48 hours of fasting significantly reduced grip strength, while administration of Dichotomine B led to a statistically significant improvement compared to the starvation group (see Table 8 and FIG. 27).

### 9-5. Measurement of Muscle Tissue Weights

To evaluate muscle loss, the weights of dissected muscle tissues-including the tibialis anterior (TA), extensor digitorum longus (EDL), gastrocnemius (GA), and soleus (SOL)-were measured post-mortem and summarized in Table 9.

TABLE 9

| | Tibialis | Extensor | | |
|---|---|---|---|---|
| | Anterior | Digitorum | Gastrocnemius | Soleus |
| | (TA) | Longus (EDL) | (GA) | (SOL) |
| Group | weight (g) | weight (g) | weight (g) | weight (g) |
| Feed | 0.061 ± 0.005 | 0.015 ± 0.003 | 0.180 ± 0.015 | 0.013 ± 0.003 |
| Feed + | 0.058 ± 0.006 | 0.012 ± 0.002 | 0.169 ± 0.014 | 0.010 ± 0.001 |
| Dichotomine B | | | | |
| Starvation | 0.054 ± 0.002** | 0.012 ± 0.003* | 0.162 ± 0.017** | 0.009 ± 0.003* |
| Starvation + | 0.058 ± 0.005^{#} | 0.014 ± 0.004 | 0.165 ± 0015 | 0.013 ± 0.005^{#} |
| Dichotomine B | | | | |

**(*p < 0.05 vs. Feed, p < 0.01 vs. Feed, ^{#}p < 0.05 vs. Starvation)

According to the above results, the decreased weight of the tibialis anterior muscle due to fasting was significantly increased by administration of Dichotomine B at a dose of 10 mg/kg (p<0.05).

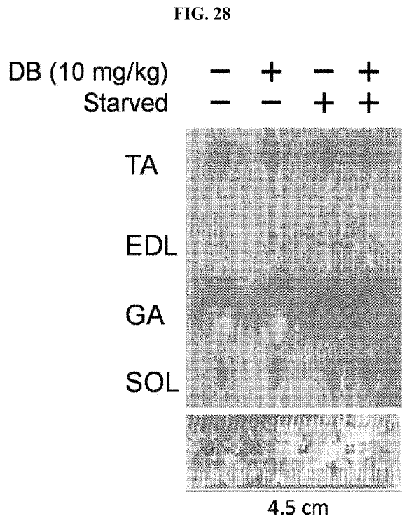

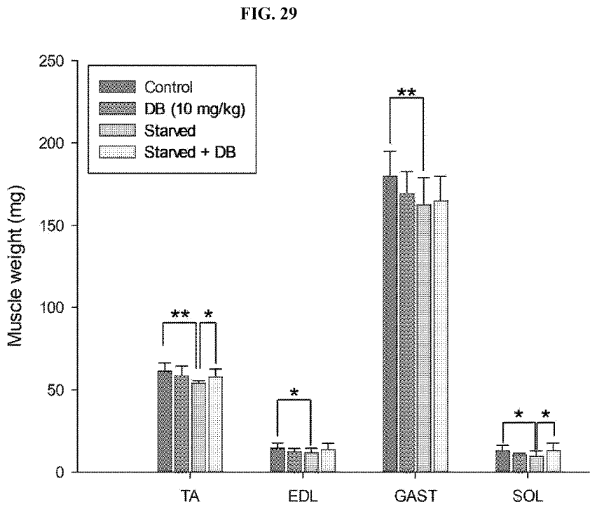

A similar improvement was also observed in the soleus muscle (p<0.05). These results confirm that Dichotomine B effectively ameliorates muscle loss caused by fasting (see Table 9 and FIGS. 28 and 29).

The present invention was accomplished by synthesizing Dichotomine B, having the above chemical structure, as a novel compound among the described substances capable of alleviating skeletal muscle atrophy.

### Example 10. Preparation of Dichotomine B

Dichotomine B can be isolated from natural sources such as Stellaria dichotoma. Alternatively, it can be chemically synthesized, and the synthesized compound may be purchased from the online stores, such as MedChemExpress (MCE).

### Structure Identification of Compound 2

¹H NMR (400 MHz, DMSO-d₆): δ ppm 3.85 (2H, d, J=7.9 Hz, H-15), 5.19 (1H, m, H-14), 7.31 (1H, t, H-6), 7.60 (1H, t, H-7), 7.75 (1H, d, J=7.5 Hz, H-8), 8.39 (1H, d, J=7.5 Hz, H-5), 8.85 (1H, s, H-4), 11.83 (1H, s, NH-9).

¹³C NMR (100 MHz, DMSO-d₆): δ ppm 66.85 (C-15), 73.90 (C-14), 113.19 (C-8), 116.63 (C-4), 120.62 (C-6), 121.15 (C-12), 122.46 (C-5), 129.13 (C-11), 129.13 (C-7) 135.31 (C-10), 135.51 (C-3), 141.66 (C-13), 145.69 (C-1), 166.82 (C-16).

MS (ESI, positive) m/z=273.2 [M+H]⁺, MS (ESI, negative) m/z=271.2 [M−H]⁻

UV (MeOH) λmax=236 nm, 269 nm

The therapeutic and preventive effects of the aforementioned Dichotomine B on skeletal muscle atrophy or sarcopenia are as described above.

### INDUSTRIAL APPLICABILITY

The present invention provides compositions that are safe, effective, and derived from natural products, which can be applied to the pharmaceutical, nutraceutical, and functional food industries for the prevention and treatment of muscle wasting conditions, including sarcopenia and cachexia.

1. A pharmaceutical composition for preventing, improving, or treating skeletal muscle atrophy or sarcopenia, comprising an extract of Stellaria dichotoma as an active ingredient.

2. The pharmaceutical composition of claim 1, wherein the extract of Stellaria dichotoma is obtained using one or more solvents selected from the group consisting of water (H₂O), C₁-C₄ lower alcohols, n-hexane, ethyl acetate, acetone, butyl acetate, 1,3-butylene glycol, methylene chloride, and mixtures thereof.

3. The pharmaceutical composition of claim 2, wherein the composition comprises a fraction obtained from the extract of Stellaria dichotoma.

4. The pharmaceutical composition of claim 1, further comprising one or more active ingredients selected from the group consisting of Dichotomine B, Glucodichotomine B, Shaftoside, Isoshaftoside, and pharmaceutically acceptable salts thereof.

5. The pharmaceutical composition of claim 4, wherein the Dichotomine B, Glucodichotomine B, Shaftoside, and Isoshaftoside are derived from the extract of Stellaria dichotoma.

6. The pharmaceutical composition of claim 1, wherein the muscle atrophy is selected from the group consisting of sarcopenia, hypotonia, disuse atrophy, mechanical unloading-induced atrophy, denervational atrophy, cachexia, drug-induced atrophy, malnutritional atrophy, and muscular dystrophy.

7. A food composition for preventing or improving skeletal muscle atrophy or sarcopenia, comprising Stellaria dichotoma extract as an active ingredient.

8. A feed composition for preventing or improving skeletal muscle atrophy or sarcopenia, comprising Stellaria dichotoma extract as an active ingredient.

9. The food composition of claim 7, further comprising one or more active ingredients selected from the group consisting of Dichotomine B, Glucodichotomine B, Shaftoside, and Isoshaftoside.

10. The food composition of claim 9, wherein the Dichotomine B, Glucodichotomine B, Shaftoside, and Isoshaftoside are derived from the Stellaria dichotoma extract.

11. The feed composition of claim 8, further comprising one or more active ingredients selected from the group consisting of Dichotomine B, Glucodichotomine B, Shaftoside, and Isoshaftoside.

12. The feed composition of claim 11, wherein the Dichotomine B, Glucodichotomine B, Shaftoside, and Isoshaftoside are derived from the Stellaria dichotoma extract.

13. The feed composition according to claim 11, wherein the Dichotomine B is chemically synthesized.
